(19) United States
(12) Patent Application Publication
(10) Pub. No.: US 2013/0218041 A1
(43) Pub. Date: Aug. 22, 2013

(54) BELLY-TYPE BREATH AUXILIARY DEVICE

(71) Applicant: Kevin LIOU, Taichung (TW)

(72) Inventor: Kevin LIOU, Taichung (TW)

(21) Appl. No.: 13/686,938

(22) Filed: Nov. 28, 2012

(30) Foreign Application Priority Data
Nov. 29, 2011 (TW) 100222484

(51) Int. Cl. A61B 5/00; A61B 5/113

(52) U.S. Cl. CPC A61B 5/486; A61B 5/113; A61B 5/72; A61B 5/7405; A61B 5/7455; A61B 5/6831; A61B 5/0004; A61B 5/4836
USPC 600/534

(57) ABSTRACT

The present invention discloses a belly-type breath auxiliary device comprising body, belt assembly and breath monitoring unit. The body includes a box. The box positions the breath monitoring unit, and connects two end of the belt assembly by elastic members. The breath monitoring unit has movement detecting unit, signal processing unit and warning unit. The movement detecting unit produces a signal when detecting the third connecting member moves to a specific position, and the signal processing unit determines the user finishing one time of effective breath by the signal. The warning unit acts to remind the user keeping his belly-type breath when the movement detecting unit does not detect the third connecting member moving to the specific position within a specific time.

### FIELD OF THE INVENTION

The present invention relates to a belly-type breath auxiliary device, and more particularly, to a device servicing users to keep effective breath by their belly.

### BACKGROUND OF THE INVENTION

Generally, people breathe by their chests or bellies. The method of breath by the force from user's belly is so called belly-type breath. The advantages of belly-type breath are: (a) strengths the function of changing air of second half of the lung, raises the vital capacity, increases the oxygen content of blood, massages viscera, strengths the toxin to get rid of, makes blood smooth in abdominal cavity, and improves health of the body; (b) through rising and falling of the belly, consumes heat and burns the fat of the body, strengths muscle group of the belly, reduces weight, and mould the body; and (c) takes breath as sport at any time.

Because most habits of breathing method of people are chest breathing method, the method of belly-type breathing method is not generally used. If there are assisting devices, they can assist people to get used to belly-type breathing easily.

A conventional breath servicing unit includes gasbag, pump and strip. The gasbag is positioned on the strip by suturing technology. The pump is attached on the strip too. The gasbag and the pump are closed on the belly of the user by binding the strip up on the body of the user. The conventional breath servicing unit assists the user to act method of belly-type breath by pumping air in and out the gasbag. But the conventional breath servicing unit has shortcomings: (a) it is passive form of use by rising and falling of the belly, it is difficult to form a habit of belly-type breath; (b) need to provide a gasbag and sew the gasbag on the strip, results in difficulties of assembly; (c) need to provide an air pumps, and make noise when the air pump fills into and takes out the air of the gasbag; and (d) the gasbag and air pump have bigger weight, increases users bear.

### SUMMARY OF THE INVENTION

The purpose of this invention is to provide a belly-type breath auxiliary device. The device comprises body, belt assembly and breath monitoring unit. The body includes a box. The box positions the breath monitoring unit, and connects two end of the belt assembly by elastic members. The breath monitoring unit has movement detecting unit, signal processing unit and warning unit. The movement detecting unit produces a signal when detecting the third connecting member moves to a specific position, and the signal processing unit determines the user finishing one time of effective breath by the signal. The warning unit acts to remind the user keeping his belly-type breath when the movement detecting unit does not detect the third connecting member moving to the specific position within a specific time.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the belly-type breath auxiliary device of the present invention;

FIG. 2 is a perspective view to show the partially assembly of the belly-type breath auxiliary of the present invention;

FIG. 3 is a perspective view to show the complete assembly of the belly-type breath auxiliary of the present invention;

FIG. 4 is a front plane view to show the status of action of the belly-type breath auxiliary of the present invention;

FIG. 5 is a top plane view to show the status of action of the belly-type breath auxiliary of the present invention;

FIG. 6 is a front plane view to show the status of action and the third connecting member 22 at the specific position of the belly-type breath auxiliary of the present invention;

FIG. 7 is a plane view to show a second embodiment of the belly-type breath auxiliary of the present invention;

FIG. 8 is a perspective view to show a third embodiment of the belly-type breath auxiliary of the present invention;

FIG. 9 is a plane view to show the third embodiment of the belly-type breath auxiliary of the present invention;

FIG. 10 is a perspective view to show a fourth embodiment of the belly-type breath auxiliary of the present invention;

FIG. 11 is a plane view to show the fourth embodiment of the belly-type breath auxiliary of the present invention;

FIG. 12 is an exploded view to show a fifth embodiment of the belly-type breath auxiliary device of the present invention;

FIG. 13 is a perspective view to show a fifth embodiment of the belly-type breath auxiliary of the present invention; and

FIG. 14 is a plane view to show the fifth embodiment of the belly-type breath auxiliary of the present invention.

### DETAILED DESCRIPTION OF THE PREFERRED EMBODIMENT

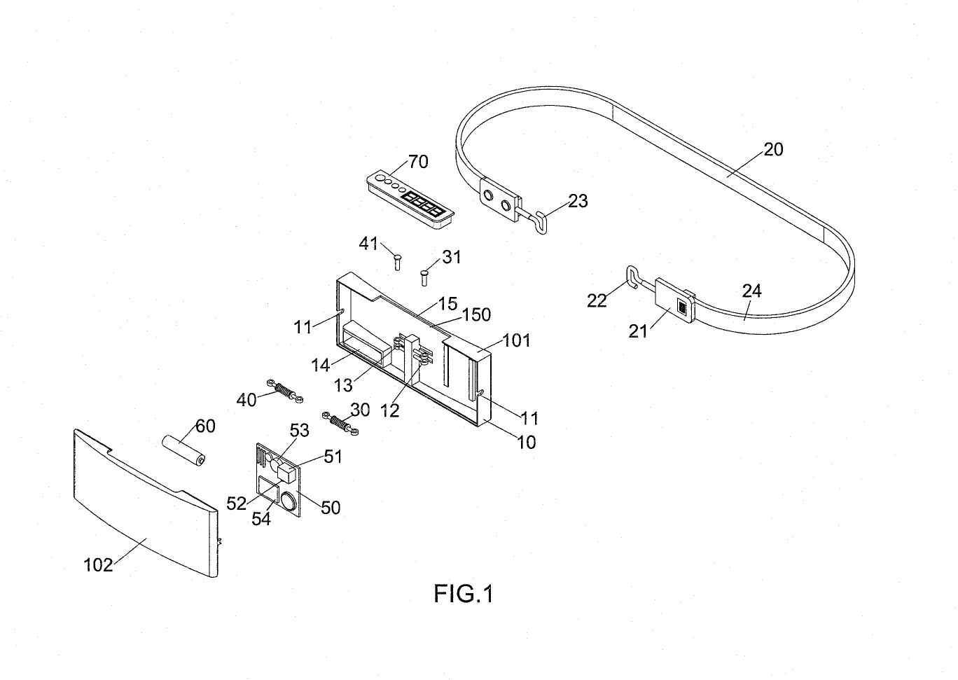

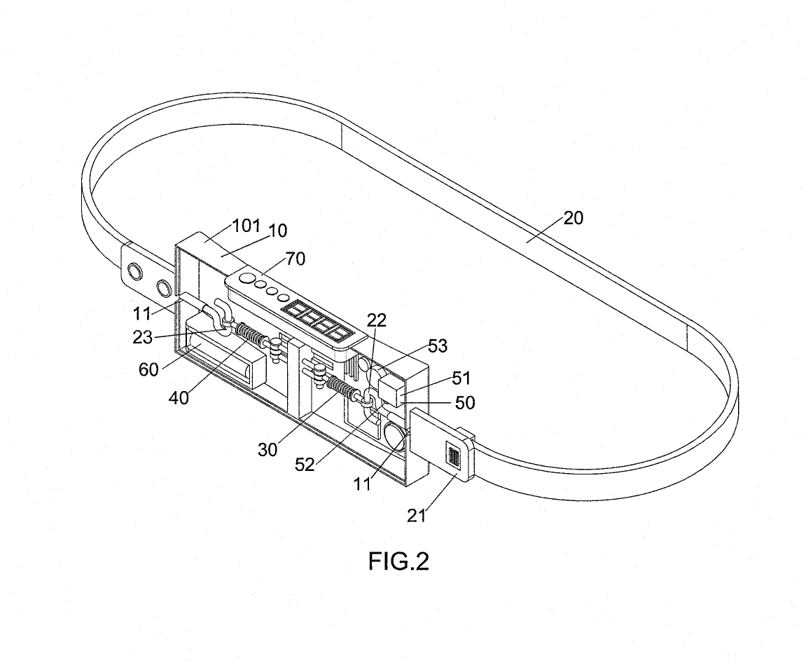

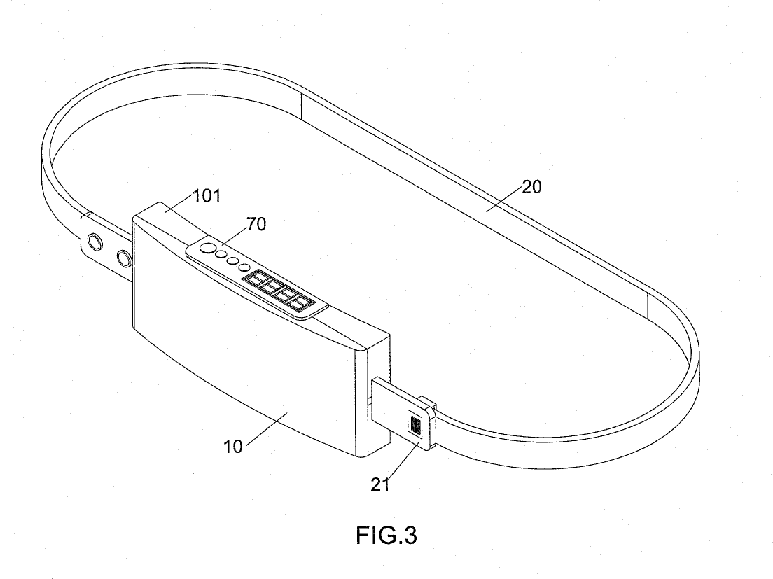

As shown in FIGS. 1 to 3, the belly-type Breath auxiliary device of the present invention includes a body 10, a belt assembly 20 and a breath monitoring unit 50. The body 10 includes a box 101 and a cover 102. A top of the box 101 is defined a first fluting 15 thereon. Two lateral sides of the box 101 are defined a trough 11 thereon respectively. An inner side of the box 101 is defined a holding trough 14 for receiving batteries 60, a first connecting member 12 and a second connecting member 13. The first fluting 15 forms a rectangular trough 150 when the cover 102 and the box 101 cover with each other. The belt assembly 20 comprises a fastener 21, a strip 24, a third connecting member 22 and a fourth connecting member 23. The fastener 21 is positioned on the strip 24 so that the strip 24 can be disassembled into several portions. The third connecting member 22 and the fourth connecting member 23 are positioned at two ends of the belt assembly 20 and penetrating one trough 11 respectively, so that the body 10 is attached on the belt assembly 20. The figure illustrates that the third connecting member 22 is fixed at one end of the fastener 21. One distal end of the third connecting member 22 and the fourth connecting member 23 are formed in hook-shaped. The belt assembly 20 is put around a belly of a user, and the body 10 is closed the belly. A first elastic member 30 with two ends connects with the first connecting member 12 and the third connecting member 22 respectively. The figure illustrates that one end of the first elastic member 30 is connected with the first connecting member 12 by a pin 31, and the other end of the first elastic member 30 is connected with the third connecting member 22 by one hook-shaped end of the third connecting member 22. A second elastic member 40 with two ends connects with the second connecting member 13 and the fourth connecting member 23 respectively. The figure illustrates that one end of the second elastic member 40 is connected with the second connecting member 13 by a pin 41, and the other end of the second elastic member 40 is connected with the fourth connecting member 23 by one hook-shaped end of the fourth connecting member 23. The breath monitoring unit 50 is positioned on the box 101 and comprises a movement detecting unit 52, a signal processing unit 51 and a warning unit 53. The breath monitoring unit 50 is supplied electric power by the batteries 60. The movement detecting unit 52 produces a signal when it detects the third connecting member 22 moving to a specific position, and the signal processing unit 51 determines the user finishing one time of effective breath according to the signal. The warning unit 53 can be a sounder, and the sounder includes a buzzer (not shown in figures). The warning unit 53 can be a vibrator. A control panel 70 is positioned at the rectangular trough 150, the control panel 70 is provided for switching on/off of the breath monitoring unit 50 and setting a monitoring mode of the breath monitoring unit 50 (such as setting a waiting time of the warning unit 53, times of breath, average time of one breath, speed of breath, manual and automatic modes. The breath monitoring unit 50 further comprises a signal transmitting unit 54. The signal transmitting unit 54 includes a USB port. The signal transmitting unit 54 can be wireless. 10.

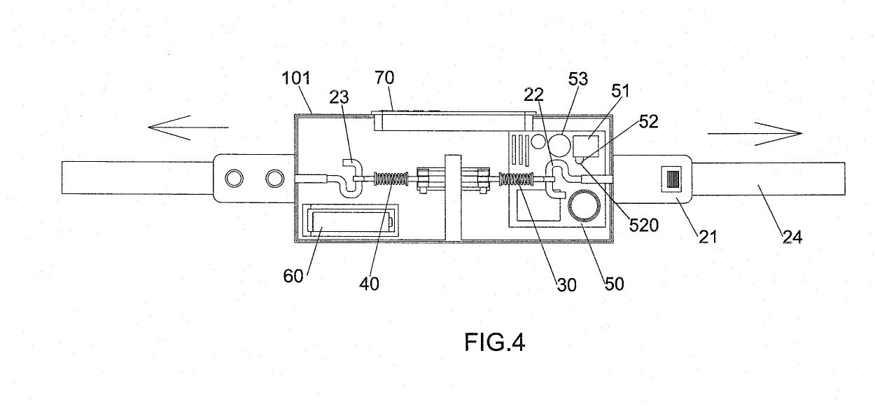

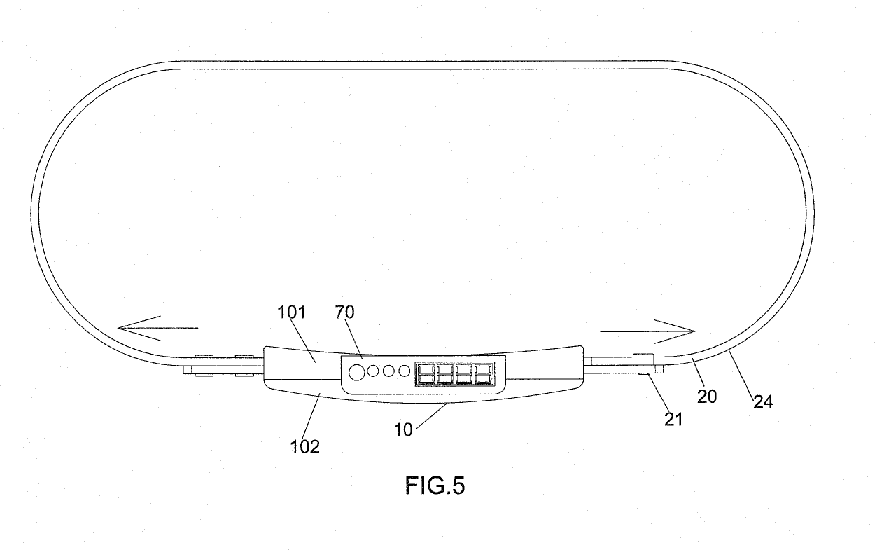

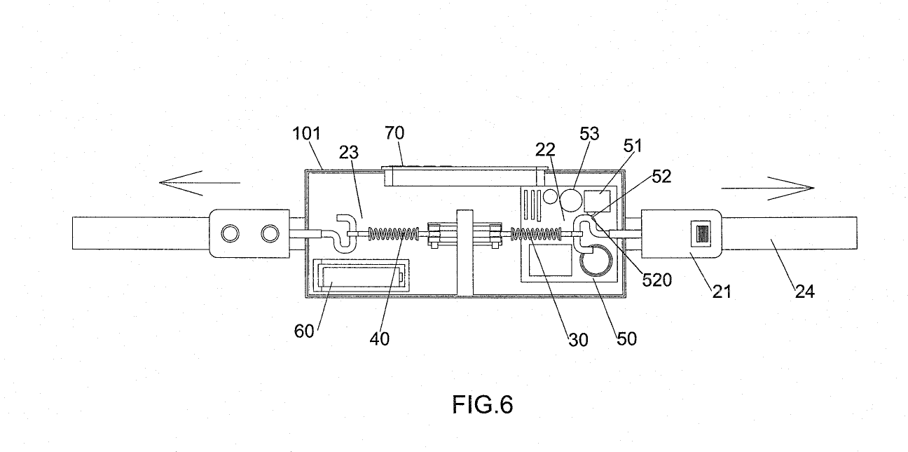

Referring to FIGS. 4 to 6, one embodiment of the present invention, the movement detecting unit 52 includes a touch switch 520. The signal is produced when the third connecting member 22 reaches the specific position and touch the touch switch 520.

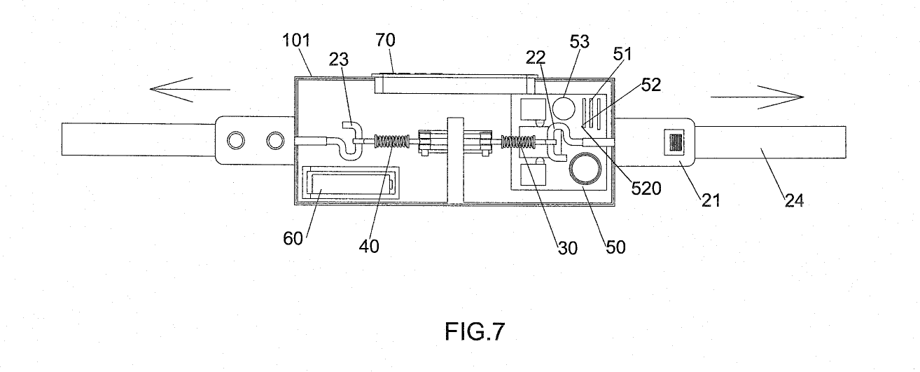

Referring to FIG. 7, one embodiment of the present invention, the movement detecting unit 52 includes an obstructing switch 521. The obstructing switch 521 includes a light emitter 522 and a light receiver 523. The signal is produced by the obstructing switch 521 when the third connecting member 22 reaches the specific position and between the light emitter 522 and the light receiver 523.

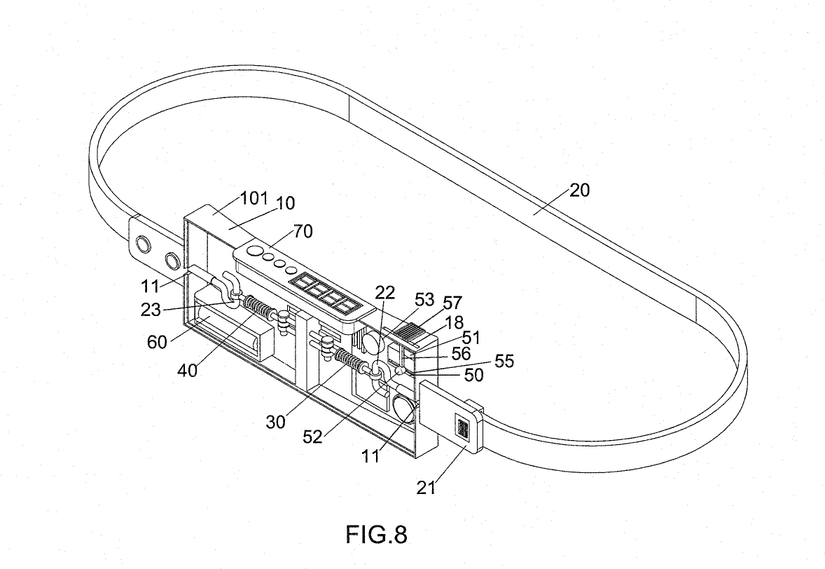

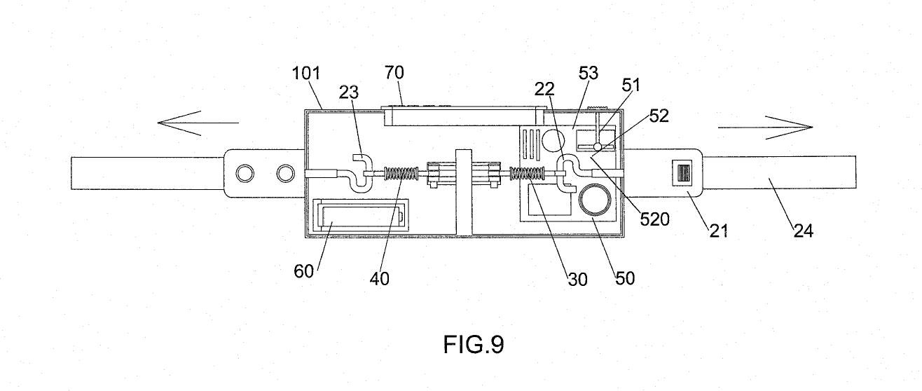

Referring to FIGS. 8 and 9, one embodiment of the present invention, the box 101 has a long trough 11 and a track 55. The movement detecting unit 52 is moveable along the track 55. The movement detecting unit 52 connects with a push button 57 by a first link 56. The first link 56 penetrates the long trough 11, and the push button 57 positions at an outer periphery of the box 101.

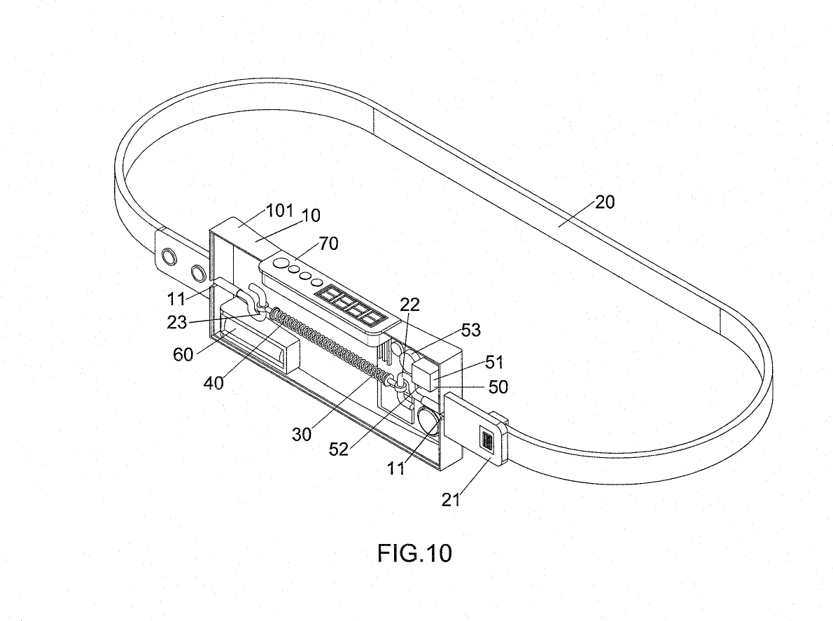

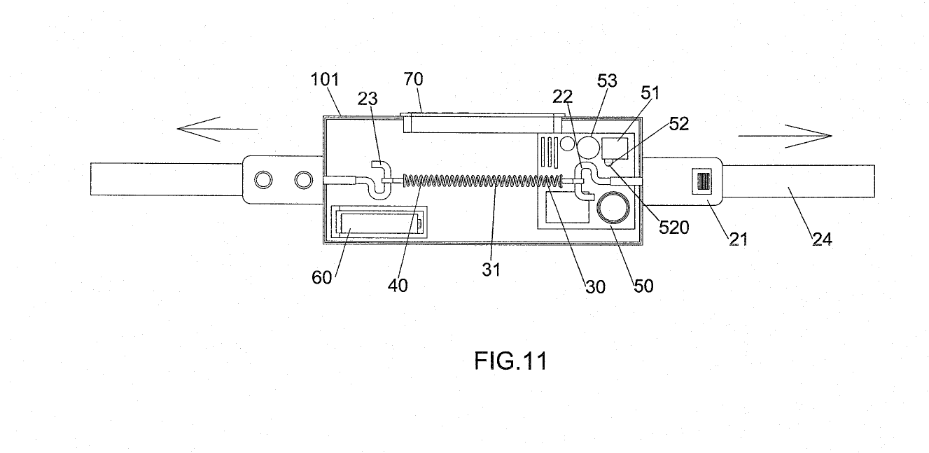

Referring to FIGS. 10 and 11, one embodiment of the present invention, the first elastic member 30 and the second elastic member 40 connects with each other to be an third elastic 31. Two ends of the third elastic 31 connect with the first connecting member 12 and the fourth connecting member 23 respectively.

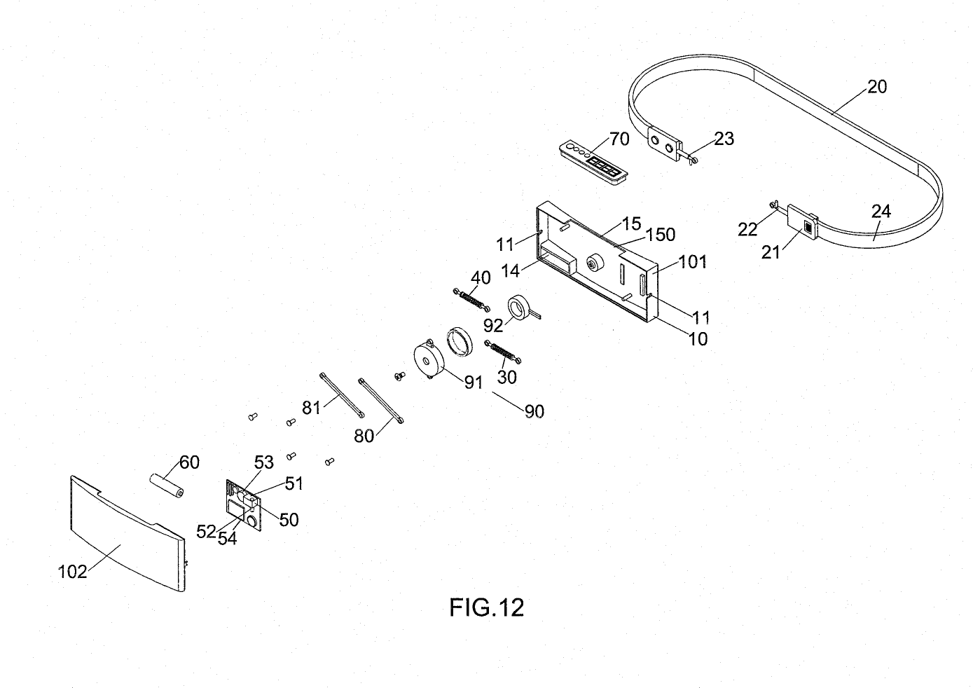

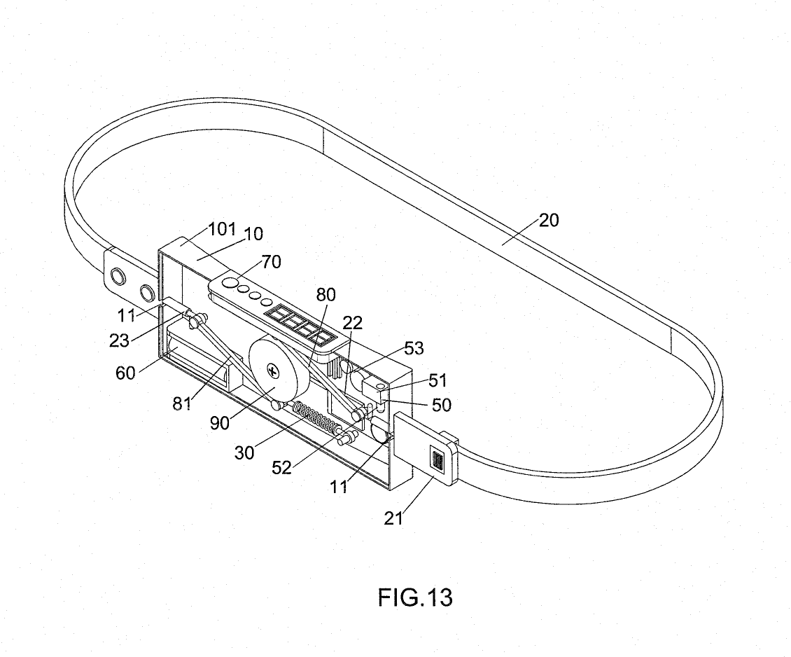

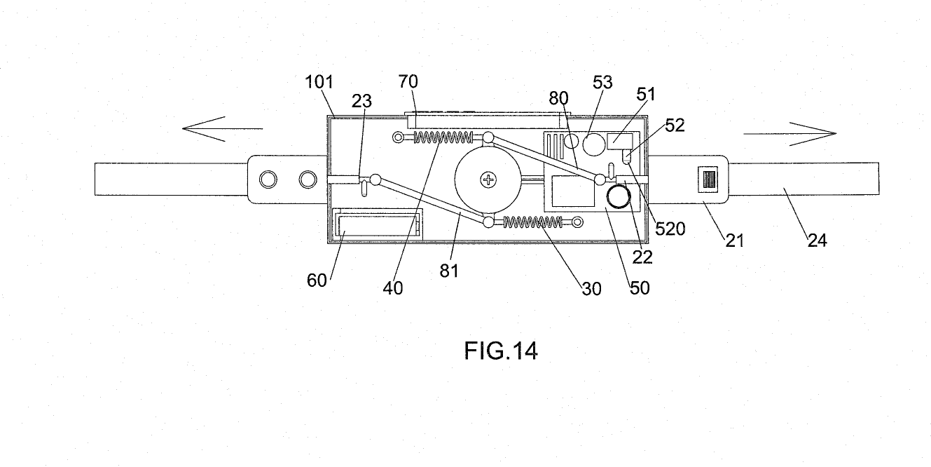

Referring to FIGS. 12 to 14, one embodiment of the present invention, the third connecting member 22 and the first elastic member 30 are pivotally connected with two ends of a second link 80 respectively. The fourth connecting member 23 and the second elastic member 40 are pivotally connected with two ends of a third link 81 respectively. The body 10 is mounted an electric power generator 90. The electric power generator 90 includes a rotor 91 and a stator 92. The stator 92 is fixed on the body 10. The second link 80 and the third link 81 are pivotally connected with peripheries of the rotor 91. When the third connecting member 22 reaches to the specific position, the rotor 91 rotates with respect to the stator 92 via the second link 80 and the third link 81 so that the electric power generator 90 produces electric power.

The advantages of the present invention are as follows: (a) user is reminded when he does not reach movement of requiring, so as to have the efficiency of independent abdominal exercise; (b) vibrates or sounds to tell users to keep continue operation, so that the result is good; (c) the whole weight of the device is light, does not bear while bind up the belly of users; and (d) has the function of setting a monitoring mode of the breath monitoring unit (such as setting a waiting time of the warning unit, times of breath, average time of one breath, speed of breath, manual and automatic modes.

While we have shown and described the embodiment in accordance with the present invention, it should be clear to those skilled in the art that further embodiments may be made without departing from the scope of the present invention.

What is claimed is:

1. A belly-type Breath auxiliary device comprising:
a body comprising a box and a cover; a top of the box being defined a first fluting thereon, two lateral sides of the box being defined a trough thereon; an inner side of the box being defined a holding trough for receiving batteries, a first connecting member and a second connecting member; the first fluting forming a rectangular trough when the cover and the box cover with each other;
a belt assembly comprising a fastener, a strip, a third connecting member and a fourth connecting member; the fastener being positioned on the strip; the third connecting member and the fourth connecting member being positioned at two ends of the belt assembly and penetrating one trough respectively, so that the body being attached on the belt assembly; the belt assembly being put around a belly of a user, and the body being closed the belly;
a first elastic member with two ends connecting with the first connecting member and the third connecting member respectively;
a second elastic member with two ends connecting with the second connecting member and the fourth connecting member respectively;
a breath monitoring unit locating on the box and comprising a movement detecting unit, a signal processing unit and a warning unit; the breath monitoring unit being supplied electric power by the batteries; the movement detecting unit producing a signal when detecting the third connecting member moving to a specific position, and the signal processing unit determining the user finishing one time of effective breath according to the signal; when the movement detecting unit having not detected the third connecting member moving to the specific position within a specific time, the warning unit acting to remind the user keeping his belly-type breath; and
a control panel being positioned at the rectangular trough, the control panel being provided for switching on/off of the breath monitoring unit, and setting a monitoring mode of the breath monitoring unit.

2. The belly-type Breath auxiliary device as claimed in claim 1, wherein the warning unit includes a sounder.

3. The belly-type Breath auxiliary device as claimed in claim 2, wherein the sounder is a buzzer.

4. The belly-type Breath auxiliary device as claimed in claim 1, wherein the warning unit includes a vibrator.

5. The belly-type Breath auxiliary device as claimed in claim 1, wherein one end of the first elastic member is connected with the first connecting member by a pin, the other end of the first elastic member is connected with the third connecting member by one hook-shaped end of the third connecting member.

6. The belly-type Breath auxiliary device as claimed in claim 1, wherein one end of the second elastic member is connected with the second connecting member by a pin, the other end of the second elastic member is connected with the fourth connecting member by one hook-shaped end of the fourth connecting member.

7. The belly-type Breath auxiliary device as claimed in claim 1, wherein the breath monitoring unit comprises a signal transmitting unit.

8. The belly-type Breath auxiliary device as claimed in claim 1, wherein the signal transmitting unit comprises a USB port.

9. The belly-type Breath auxiliary device as claimed in claim 1, wherein the signal transmitting unit is wireless.

10. The belly-type Breath auxiliary device as claimed in claim 1, wherein the movement detecting unit includes a touch switch; the signal is produced when the third connecting member reaches the specific position and touch the touch switch.

11. The belly-type Breath auxiliary device as claimed in claim 1, wherein the movement detecting unit includes an obstructing switch; the obstructing switch includes a light emitter and a light receiver; the signal is produced by the obstructing switch when the third connecting member reaches the specific position and between the light emitter and the light receiver.

12. The belly-type Breath auxiliary device as claimed in claim 1, wherein the box has a long trough and a track, the movement detecting unit is moveable along the track; the movement detecting unit connects with a push button by a first link; the first link penetrates the long trough, and the push button positions at an outer periphery of the box.

13. The belly-type Breath auxiliary device as claimed in claim 1, wherein the third connecting member and the first elastic member are pivotally connected with two ends of a second link respectively; the fourth connecting member and the second elastic member are pivotally connected with two ends of a third link respectively; the body is mounted an electric power generator; the electric power generator includes a rotor and a stator; the stator is fixed on the body; the second link and the third link are pivotally connected with peripheries of the rotor; when the third connecting member reaches to the specific position, the rotor rotates with respect to the stator via the second link and the third link so that the electric power generator produces electric power.
